(19) United States
(12) Patent Application Publication
(10) Pub. No.: US 2025/0368683 A1
(43) Pub. Date: Dec. 4, 2025

(54) METHOD FOR TREATING OR PREVENTING BRONCHIAL ALLERGIC DISEASES WITH PEPTIDE COMPOSITION

(71) Applicant: Greenyn Biotechnology Co., Ltd, Taichung City (TW)

(72) Inventors: Pang-Kuei Hsu, Taichung City (TW); Chih-Kuo Kao, Taichung City (TW); Yu-Cheng Lin, Taichung City (TW)

(21) Appl. No.: 19/219,438

(22) Filed: May 27, 2025

Related U.S. Application Data
(60) Provisional application No. 63/653,541, filed on May 30, 2024.

(51) Int. Cl. C07K 7/06; A61K 38/00; A61P 11/06; C07K 7/08

(52) U.S. Cl. CPC C07K 7/06; A61P 11/06; C07K 7/08; A61K 38/00

(57) ABSTRACT

A method for treating or preventing a bronchial allergic disease with a peptide composition includes administering an effective amount of the peptide composition to an individual in need of the peptide composition so as to improve or prevent asthma or another bronchial allergic disease. The peptide composition has at least one polypeptide, and the amino acid sequence of the polypeptide is represented by one of SEQ ID No. 1 to SEQ ID No. 10.

### CROSS-REFERENCE TO RELATED APPLICATIONS

This application claims the benefit of U.S. Provisional Patent Application No. 63/653,541, which was filed on May 30, 2024.

### REFERENCE TO AN ELECTRONIC SEQUENCE LISTING

The content of the electronic sequence listing (SL.xml; Size: 9,590_bytes; and Date of Creation: May 22, 2025) is herein incorporated by reference in its entirety.

### FIELD OF THE INVENTION

The present invention relates to a use of a functional peptide. More particularly, the invention relates to a method for treating or preventing a bronchial allergic disease with a peptide composition.

### DESCRIPTION OF THE RELATED ART

Bronchial allergy, also known as airway hyperresponsiveness, refers to allergic reactions of the trachea caused by allergen stimulation and may give rise to symptoms such as inflammation, swelling, or narrowing of the airways and phlegm accumulation in the airways, resulting in difficulty in breathing in severe cases. Common allergens include dust mites, animal hair, airborne dust, powder, and pollen, among others. Asthma is a disease, or airway allergy, caused by chronic inflammation of the bronchi, and some of the clinical symptoms of asthma are recurrent coughing, wheezing, shortness of breath, chest tightness, and difficulty in breathing.

According to statistics, about 300 million people worldwide suffer from asthma, and about 180 thousand people die of asthma per year. Clinically, patients with asthma, which is difficult to cure, are treated mainly by medication. Drugs for asthma can be generally divided into those for symptom control and those for use in the acute phase. Some examples of the former are steroids for inhalation and steroids for oral administration, and an example of the latter is bronchodilators. However, studies have shown that taking oral steroids for a long time may have such side effects as osteoporosis and a moon face; that a bronchodilator may cause palpitations, shaking of the hands, etc. as its side effects; and that using a bronchodilator as a drug for daily control not only fails to control asthma, but also increases the risk of death.

### SUMMARY OF THE INVENTION

The primary objective of the present invention is to provide a method for treating or preventing a bronchial allergic disease with a peptide composition, wherein the method uses a polypeptide having the activity to inhibit interleukin-4 (IL-4) and/or IL-13 interleukin-13 (IL-13) and includes administering the polypeptide to an individual to effectively improve bronchial allergy or a disease caused by bronchial allergy, thereby producing the effect of daily health maintenance and allergy symptom amelioration.

Another objective of the present invention is to provide a method for treating or preventing a bronchial allergic disease with a peptide composition, wherein the peptide composition can effectively regulate an individual's immune responses and can therefore serve as a means for daily health maintenance to reduce a patient's use of drugs or frequency of drug use.

To achieve the foregoing objectives, the present invention provides a method for treating or preventing a bronchial allergic disease with a peptide composition, wherein the method includes administering an effective amount of the peptide composition to an individual in need of the peptide composition, and wherein the peptide composition has at least one polypeptide whose amino acid sequence is represented by one of SEQ ID No. 1 to SEQ ID No. 10.

The peptide composition may be a food, a nutritional supplement, or a drug.

More specifically, the peptide composition disclosed herein contains a polypeptide having the activity to inhibit IL-4 and/or IL-13 and to reduce the concentration of immunoglobulin E (IgE). Therefore, administering the peptide composition to an individual, especially an individual suffering from, or having a high risk of developing, an allergen-induced bronchial allergic disease such as asthma, can effectively improve or alleviate the bronchial allergic disease.

In one embodiment of the present invention, the peptide composition contains at least four polypeptides, namely the polypeptides represented by SEQ ID No. 4, SEQ ID No. 6, SEQ ID No. 8, and SEQ ID No. 9.

In another embodiment of the present invention, the peptide composition contains the polypeptides represented by SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, and SEQ ID No. 10.

### BRIEF DESCRIPTION OF DRAWINGS

FIG. 1 shows the test result of the IL-4 inhibiting effect of each polypeptide under test.

FIG. 2 shows the test result of the airway resistance (Penh) value of each group of mice.

FIG. 3 shows the calculation result of the area under the curve in FIG. 2 for each group of mice.

FIG. 4 shows the analysis result of the splenocyte proliferation activity of each group of mice.

FIG. 5 shows the analysis result of the natural killer cell activity of each group of mice.

FIG. 6 shows the analysis result of the IL-13 concentration in the BALF of each group of mice.

FIG. 7 shows the analysis result of the IL-4 concentration in the BALF of each group of mice.

FIG. 8 shows the analysis result of the IFN-γ concentration in the BALF of each group of mice.

FIG. 9 shows the analysis result of the serum IgE concentration of each group of mice.

FIG. 10 shows the analysis result of the serum IgG1 concentration of each group of mice.

### DETAILED DESCRIPTION OF THE INVENTION

The present invention discloses a method for treating or preventing a bronchial allergic disease with a peptide composition. The method includes administering an effective amount of the peptide composition to an individual in need of the peptide composition so as to improve or prevent asthma or another bronchial allergic disease. The peptide composition has at least one polypeptide, and the amino acid sequence of the polypeptide is represented by one of SEQ ID No. 1 to SEQ ID No. 10.

More specifically, the polypeptides disclosed herein, i.e., the polypeptides represented by SEQ ID No. 1 to SEQ ID No. 10, have the activity to reduce IL-4, IL-13, and an imbalance of T helper 2 (Th2) cells. Therefore, administering the peptide composition disclosed herein to an individual can effectively treat or improve a bronchial allergic disease such as asthma or allergic coughing.

It should be pointed out that IL-4 and IL-13 are cytokines that jointly participate in the maintenance and exacerbation of chronic bronchial inflammation. Accordingly, a substance having IL-4 and IL-13 inhibitory activity, such as any of the polypeptides disclosed herein or a composition containing any of the polypeptides disclosed herein, can cut off signaling downstream of IL-4 and IL-13 effectively, inhibit inflammation and airway hyperresponsiveness, and thereby alleviate or improve the clinical symptoms of bronchial allergy. For example, dupilumab, a drug clinically used for asthma nowadays, is an IL-4Rα antagonist that improves the bronchial function and inflammatory indicators in patients with moderate-to-severe asthma by way of IL-4 and IL-13 inhibition.

The polypeptides represented by SEQ ID No. 1 to SEQ ID No. 10 are shown in Table 1 below. Each uppercase letter in the amino acid sequences in Table 1 represents one amino acid, and unless otherwise stated, the amino acid represented by each uppercase letter should be understood and explained according to general common knowledge in the field to which the present invention pertains.

TABLE 1
Polypeptide sequences

| Sequence no. | Amino acid sequence |
|---|---|
| SEQ ID No. 1 | INFVTEEDKR |
| SEQ ID No. 2 | LGVTNINISDP |
| SEQ ID No. 3 | RLTDYFTID |
| SEQ ID No. 4 | SMNLSLVLQVV |
| SEQ ID No. 5 | ALYPLLTTLTD |
| SEQ ID No. 6 | VRYLEWSNIESIIA |
| SEQ ID No. 7 | LVGTDVATAQAP |
| SEQ ID No. 8 | GHCGCSSG |
| SEQ ID No. 9 | NFIVTGLQDIM |
| SEQ ID No. 10 | IGVDEHYTPI |

In one embodiment of the present invention, the peptide composition contains the peptide represented by SEQ ID No. 4, SEQ ID No. 6, SEQ ID No. 8, or SEQ ID No. 9.

In another embodiment of the present invention, the peptide composition contains the peptides represented by SEQ ID No. 4, SEQ ID No. 6, SEQ ID No. 8, and SEQ ID No. 9.

In yet another embodiment of the present invention, the peptide composition contains the polypeptides represented by SEQ ID No. 1 to SEQ ID No. 10.

In still another embodiment of the present invention, the polypeptides are prepared by artificial synthesis or by a microbial production platform.

In a further embodiment of the present invention, the polypeptides are derived from animal milk, such as a cow's milk, a goat's milk, or a mare's milk, by separation, extraction, and/or purification. For example, the polypeptides are separated from a goat's milk by an extraction method, and applicable extraction methods include solvent extraction, supercritical fluid extraction, and other extraction methods well known to a person of ordinary skill in the art. Taking solvent extraction for example, the extraction solvent to be used may be pure water, a lower alcohol, acetone, ethyl acetate, or a combination of any two of the above solvents, and when multiple extraction solvents are used, extraction may be performed sequentially with the different extraction solvents based on their different solubilities.

The term “polypeptide” refers to a linear molecule or ring-shaped molecule containing two or more amino acids. A polypeptide may be separated from a substance of a natural source or be prepared artificially, such as by chemical synthesis or by a recombinant microbial production platform.

The term “composition” refers to a mixture of two or more ingredients, wherein the ingredients include but are not limited to an active ingredient, an excipient, a solvent, a carrier, a buffer, and other ingredients that are pharmaceutically acceptable or are acceptable in food. A composition may be in a solid state, a liquid state, a gaseous state, a gel state, an emulsion state, or any other dosage form and may be used for disease treatment, disease prevention, or daily health maintenance. The composition in the present invention may be a pharmaceutical, a food, or a nutritional supplement.

The term “effective amount” refers to a dose/dosage capable of producing the expected biological effect or therapeutic effect, and the aforesaid effects include but are not limited to disease prevention, symptom alleviation, improvement of physiological parameters, and delay of disease progression. The effective amount in which a substance is administered to an individual can be adjusted according to such factors as the individual's age, body weight, and disease condition and the method and frequency of administration and be determined according to clinical or preliminary trial data.

The term “active ingredient” refers to an ingredient that is part of a composition, that has the expected biological activity, and that can directly or indirectly induce the expected effect of treating, preventing, diagnosing, alleviating, improving, or controlling a disease. Generally, an active ingredient makes up 0.001-100% of the total weight of a composition. An active ingredient may be a substance of a natural source or a partially or completely synthetic substance. In the present invention, for example, the active ingredient is at least one polypeptide.

The term “administer” refers to introducing an active ingredient or the composition containing the active ingredient into a living organism through an acceptable route, wherein acceptable routes include but are not limited to oral delivery, injection (intravenous, subcutaneous, or intramuscular), inhalation, transdermal delivery, rectal delivery, and other medically acceptable routes, the objective being to produce the expected biological effect or therapeutic effect in the living organism.

The term “bronchial allergy” refers to a physiological state where the bronchi show an exaggerated response to such a stimulus as cold air, an allergen, or a chemical substance. Bronchial allergy typically manifests as reversible airway constriction, airflow limitation, or a tendency toward an asthma attack.

The term “asthma” refers to a chronic inflammatory disease of the respiratory tract that is characterized by at least one of the following symptoms: reversible airway obstruction, bronchial hyperresponsiveness, shortness of breath, wheezing, and coughing.

The term “a” and “at least one” refer to the quantity of one or more than one. Unless there is explicit limitation in the context, the terms shall encompass cases where there is a single unit or multiple units of the noun identified by either of the terms.

The term “IL-4 (interleukin-4)” refers to a key cytokine released by the Th2 cell. The cytokine plays a critical role in asthma and bronchial allergic inflammation and participates in the production of IgE.

The term “IL-13 (interleukin-13)” refers to another key cytokine released by the Th2 cell. This cytokine also plays a critical role in asthma and bronchial allergic inflammation and can promote airway epithelial remodeling, mucus secretion, and bronchial smooth muscle hyperresponsiveness.

To demonstrate the technical features of the present invention and their effects, some embodiments are detailed below with reference to the accompanying drawings.

The dose of the composition used in the animal test in the following examples can be changed, depending on such factors as the individuals to which the composition is administered, the dosage form of the composition, and the type of the composition. Dose changes can be calculated by conversion according to general common knowledge in the technical field to which the present invention pertains. For example, the composition disclosed herein is administered to a mouse at a dose of 45.05 mg/kg, and the equivalent dose for an adult human weighing 60 kg is 3.65 mg/kg (about 219 mg/day).

### Example 1: Preparation of Polypeptides

The polypeptides represented by SEQ ID No. 1-10 in Table 1 were prepared by artificial synthesis, and the amino acid sequence of each polypeptide has been verified.

### Example 2: Cell Test

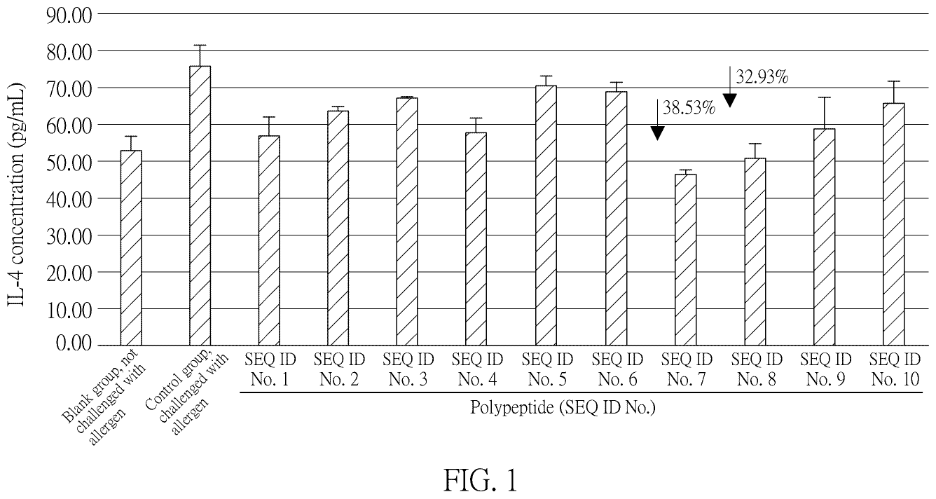

In this example, the cell line used was the P815 mouse mastocytoma cell line, and the allergen was compound 48/80. To start with, each of the polypeptides represented by SEQ ID No. 1-10 was co-cultured with the P815 cells for 20 minutes, was then added with compound 48/80, and then continued co-culturing for a total of 6 hours. When the culturing process was completed, the supernatants were collected, and the amounts of IL-4 and IL-13 secretions were analyzed by enzyme-linked immunosorbent assay (ELISA), with the results shown in FIG. 1 and Table 2.

TABLE 2
Each polypeptide's IL-4 and IL-13 inhibitory activity

| Sequence no. | IL-4 inhibitory activity | IL-13 inhibitory activity |
|---|---|---|
| SEQ ID No. 1 | −25.2 | 0.0 |
| SEQ ID No. 2 | −16.0 | 0.0 |
| SEQ ID No. 3 | −8.9 | 0.0 |
| SEQ ID No. 4 | −32.9 | −21.4 |
| SEQ ID No. 5 | −13.1 | −71.4 |
| SEQ ID No. 6 | −22.2 | −60.7 |
| SEQ ID No. 7 | −23.9 | 0.0 |
| SEQ ID No. 8 | −38.5 | 0.0 |
| SEQ ID No. 9 | −11.5 | −57.1 |
| SEQ ID No. 10 | −7.2 | 0.0 |

It can be known from the results in FIG. 1 and Table 2 that the polypeptides represented by SEQ ID No. 1-10 had IL-4 inhibitory activity; that the polypeptides represented by SEQ ID NO. 4, SEQ ID No. 5, and SEQ ID No. 9 had IL-13 inhibitory activity; and that all in all, the polypeptides represented by SEQ ID NO. 4, SEQ ID No. 6, SEQ ID No. 8, and SEQ ID No. 9 showed relatively good inhibitory activity on the Th2 pathway.

It can be inferred from the above that the polypeptides disclosed herein, i.e., the polypeptides represented by SEQ ID No. 1-10, can improve inhaled allergic reactions (e.g., bronchial allergy, asthma, bronchitis, and other similar diseases) by reducing the increase in IL-4 and/or IL-13 level caused by allergic responses, and by decreasing the rise in allergic phenomena skewed toward a Th2-dominant bias. In other words, administering an effective amount of any of the polypeptides represented by SEQ ID No. 1-10 to an individual suffering from bronchial allergy or a disease related thereto can effectively improve or alleviate the symptoms of bronchial allergy, with the administration of the polypeptide represented by SEQ ID No. 1, SEQ ID No. 6, SEQ ID NO. 8, or SEQ ID No. 9 producing a relatively good improving effect.

### Example 3: Animal Test

Mice were randomly divided into three groups. The test lasted for 23 days. Each group of mice was treated as follows:
Group 1 of mice (blank group): normal mice on which no sensitization treatment was performed and to which the peptide composition disclosed herein was not administered
Group 2 of mice (asthma group): asthmatic-model mice that received a challenge treatment from day 16 to day 22 of the test
Group 3 of mice (medicated group): asthmatic-model mice that received a challenge treatment from day 16 to day 22 of the test and to which the peptide composition disclosed herein was administered at a dose of 45.05 mg/kg body weight (B.W.) on a daily basis throughout the test

The asthmatic models were established in the following manner. On days 1, 8, and 15 of the test, ovalbumin (OVA) was administered to each mouse intended as an asthmatic model to induce sensitization, causing inflammation in the respiratory tract of the mouse.

The challenge treatment was performed in the following manner. From day 16 to day 22 of the test, a PM 2.5 sample was administered daily at a dose of 15 μg/mouse to each mouse intended to receive the treatment. The PM 2.5 sample was standard suspended particulate matter collected from an urban environment.

The active ingredients of the peptide composition used in the test included the polypeptides represented by SEQ ID No. 1-10, and the polypeptides were combined in equal or practically equal ratios.

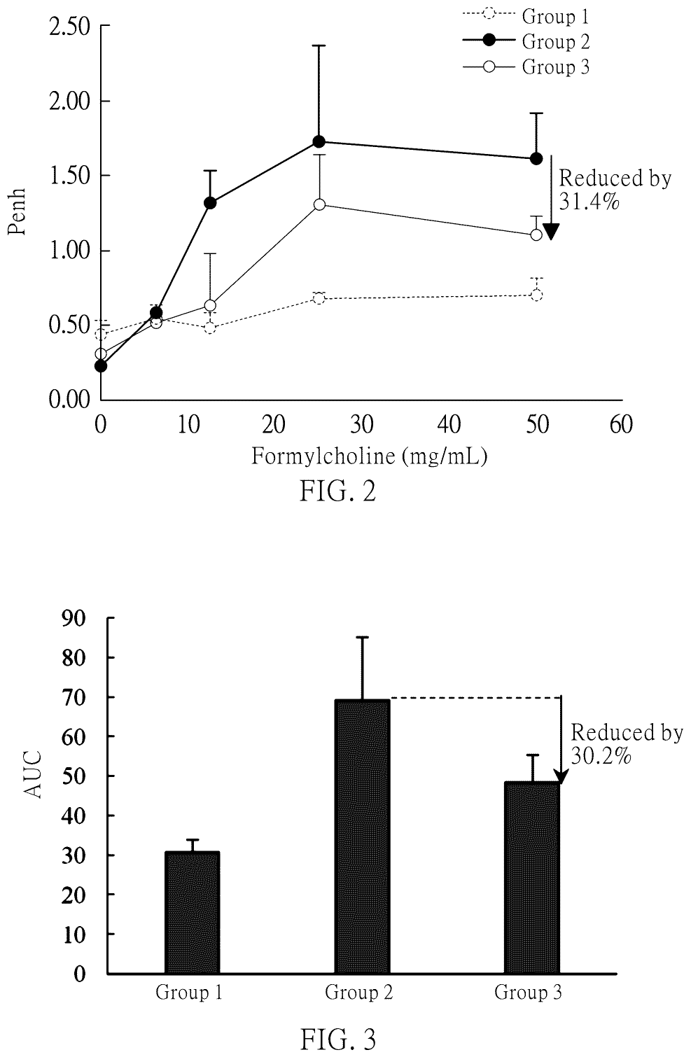

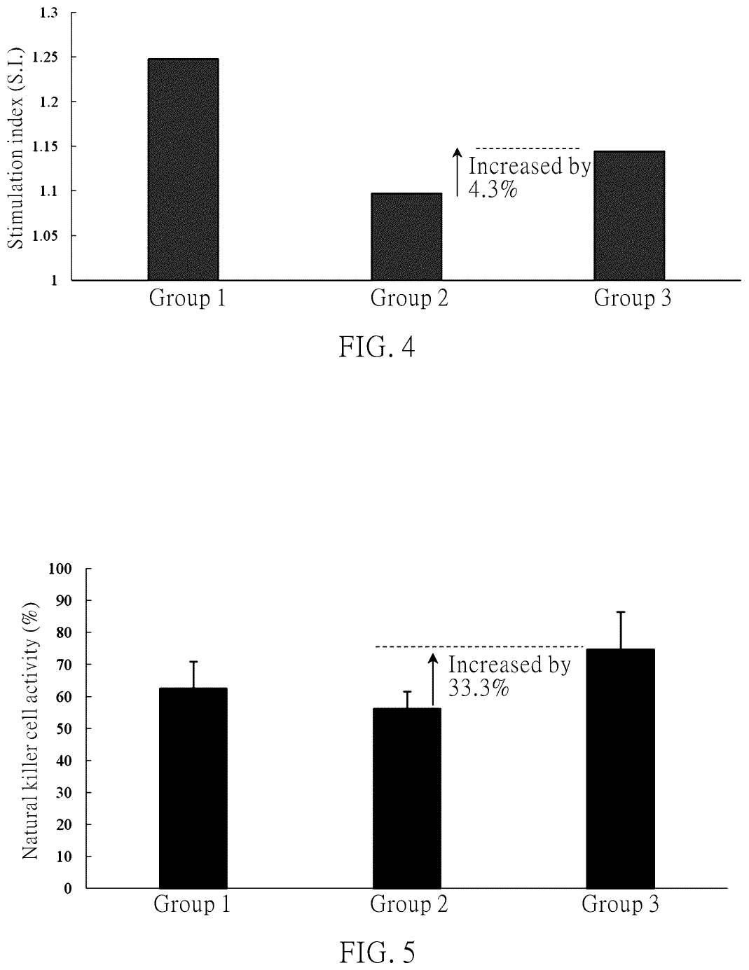

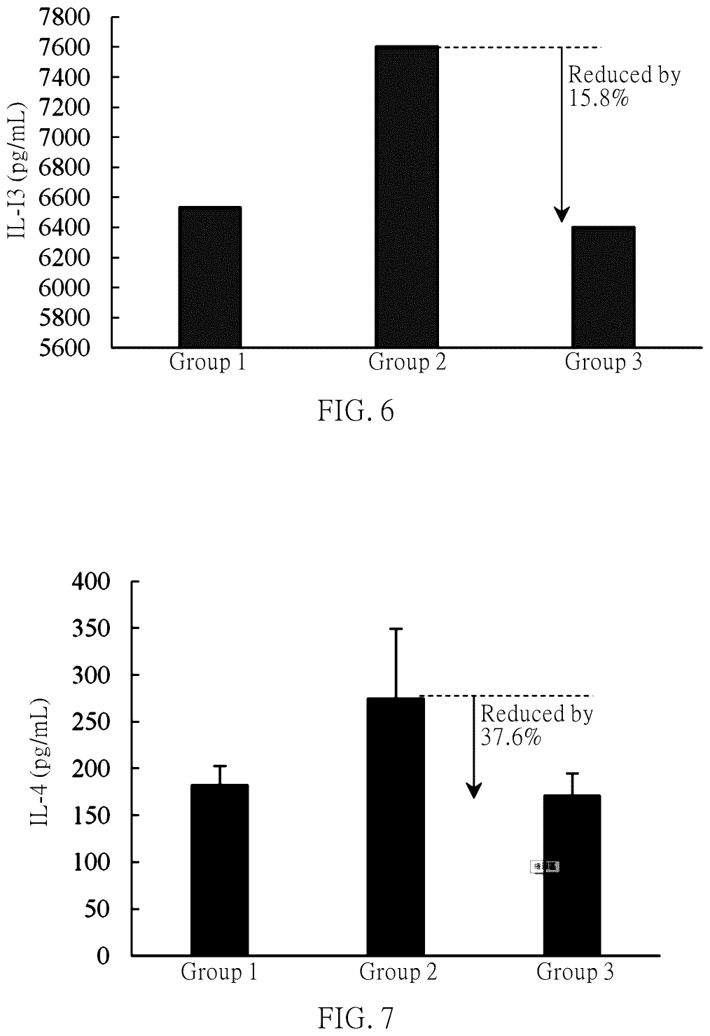

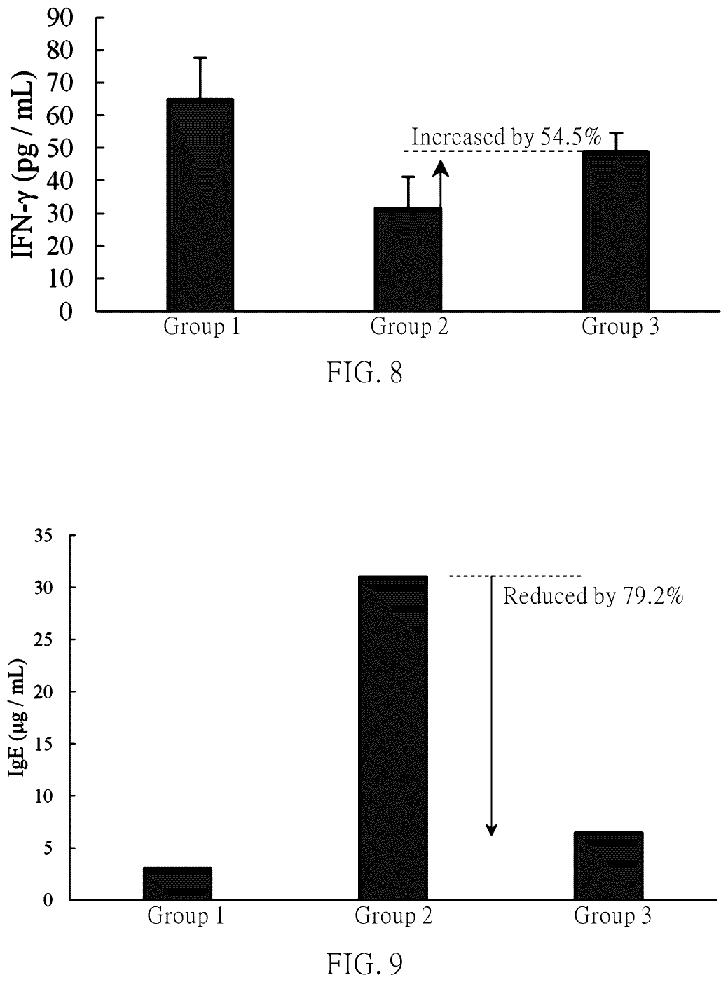

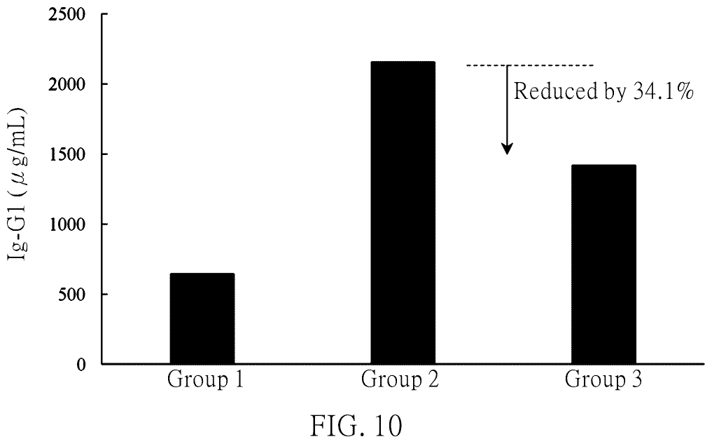

At the end of the test, the airway resistance (Penh) value of each group of mice was measured with a whole-body plethysmograph, and the areas under the time-concentration curve (AUC) were calculated, with the results shown in FIG. 2 and FIG. 3. The splenocytes of each group of mice were collected, and the splenocyte proliferation activity and natural killer cell (NK cell) activity of each group of mice were determined, with the results shown in FIG. 4 and FIG. 5. The bronchoalveolar lavage fluid (BALF) of each group of mice was collected, and the IL-13, IL-4, and interferon-γ (IFN-γ) concentrations in the BALF of each group of mice were measured by ELISA, with the results shown in FIG. 6 to FIG. 8. In addition, the IgE concentration and immunoglobulin G subclass-1 (IgG1) concentration in the serum of each group of mice were analyzed, with the results shown in FIG. 9 and FIG. 10.

It can be known from the results in FIG. 2 and FIG. 3 that the airway resistance of group 3 of mice was significantly lower than that of group 2 of mice by more than 30%. This indicates that the peptide composition disclosed herein can effectively improve, i.e., alleviate, the symptoms of asthma.

It can be known from the results in FIG. 4 and FIG. 5 that the splenocyte proliferation activity and NK cell activity of group 3 of mice were both higher than those of group 2 of mice. This indicates that after the administration of the peptide composition disclosed herein, the splenocytes were activated in a regulated manner, and a balance between T helper 1 (Th1) cells and Th2 cells was achieved. In other words, the results in FIGS. 4 and FIG. 5 show that the peptide composition disclosed herein helps improve allergic asthma, in particular the Th2 hyperactivation type.

The results in FIG. 6 to FIG. 8 show that the IL-4 and IL-13 concentrations of group 3 of mice were lower than those of group 2 of mice by 37.6% and 15.8% respectively, and that the IFN-γ concentration in the BALF of group 3 of mice was higher than that of group 2 of mice by 54.5%. Moreover, it can be known from the results in FIG. 9 and FIG. 10 that the serum IgE concentration and serum IgG1 concentration of group 3 of mice were lower than those of group 2 of mice by 79.2% and 34.1% respectively. The results in FIG. 6 to FIG. 10 show that the peptide composition disclosed herein successfully regulated the expression of bronchial inflammatory cytokines, lowered the IL-4 and IL-13 concentrations, increased the IFN-γ concentration, and caused a significant reduction in the serum IgE concentration. This indicates that the peptide composition disclosed herein does have the effect of preventing, or reducing the onset of, allergic asthma and avoiding immune imbalance.

According to the above, the polypeptides disclosed herein, i.e., the polypeptides represented by SEQ ID No. 1-10, have IL-4 and/or IL-13 inhibitory activity. Therefore, administering an effective amount of the polypeptides disclosed herein or a composition containing the polypeptides to an individual suffering from, or at high risk of developing, the symptoms of bronchial allergy can effectively prevent or improve diseases induced by bronchial allergy, such as asthma.

What is claimed is:

1. A method for treating or preventing a bronchial allergic disease, comprising the step of administering an effective amount of a peptide composition to an individual in need of the peptide composition, wherein the peptide composition has at least one polypeptide, and the polypeptide has an amino acid sequence represented by one of SEQ ID No. 1 to SEQ ID No. 10.

2. The method for treating or preventing a bronchial allergic disease as claimed in claim 1, wherein the peptide composition comprises a plurality of said polypeptides, and the polypeptides are represented by SEQ ID No. 4, SEQ ID No. 6, SEQ ID No. 8, and SEQ ID No. 9.

3. The method for treating or preventing a bronchial allergic disease as claimed in claim 1, wherein the peptide composition comprises a plurality of said polypeptides, and the polypeptides are represented by SEQ ID No. 1, SEQ ID No.2, SEQ ID No. 3, SEQ ID No. 4, SEQ ID No. 5, SEQ ID No. 6, SEQ ID No. 7, SEQ ID No. 8, SEQ ID No. 9, and SEQ ID No. 10.

4. The method for treating or preventing a bronchial allergic disease as claimed in claim 1, wherein the peptide composition is a food, a nutritional supplement, or a drug.

5. The method for treating or preventing a bronchial allergic disease as claimed in claim 1, wherein the peptide composition is used to inhibit activity of interleukin-4 (IL-4) and/or interleukin-13 (IL-13) in the individual's cells.

6. The method for treating or preventing a bronchial allergic disease as claimed in claim 3, wherein the peptide composition is used to reduce the individual's airway resistance.

7. The method for treating or preventing a bronchial allergic disease as claimed in claim 3, wherein the peptide composition is used to reduce the individual's serum immunoglobulin E (IgE) concentration.

8. The method for treating or preventing a bronchial allergic disease as claimed in claim 1, wherein the bronchial allergic disease is asthma.


SEQUENCE LISTING (10 sequences)

SEQ ID NO: 1 - protein, 10 aa, synthetic construct
INFVTEEDKR

SEQ ID NO: 2 - protein, 11 aa, synthetic construct
LGVTNINISDP

SEQ ID NO: 3 - protein, 9 aa, synthetic construct
RLTDYFTID

SEQ ID NO: 4 - protein, 11 aa, synthetic construct
SMNLSLVLQVV

SEQ ID NO: 5 - protein, 11 aa, synthetic construct
ALYPLLTTLTD

SEQ ID NO: 6 - protein, 14 aa, synthetic construct
VRYLEWSNIESIIA

SEQ ID NO: 7 - protein, 12 aa, synthetic construct
LVGTDVATAQAP

SEQ ID NO: 8 - protein, 8 aa, synthetic construct
GHCGCSSG

SEQ ID NO: 9 - protein, 11 aa, synthetic construct
NFIVTGLQDIM

SEQ ID NO: 10 - protein, 10 aa, synthetic construct
IGVDEHYTPI